(19) United States
(12) Patent Application Publication
(10) Pub. No.: US 2026/0315468 A1
(43) Pub. Date: Oct. 15, 2026

(54) PHARMACEUTICAL COMPOSITION AND USE THEREOF

(71) Applicant: SHANGHAI ALLIST PHARMACEUTICAL CO., LTD., Shanghai (CN)

(72) Inventors: Huibing Luo, Shanghai (CN); Qing Li, Shanghai (CN); Jerry Hsu, San Mateo, CA (US); Luna Musib, Danville, CA (US); Stuart Lutzker, Walnut Creek, CA (US); Marcin Kowanetz, San Francisco, CA (US)

(21) Appl. No.: 19/108,540

(22) PCT Filed: Sep. 19, 2023
(86) PCT No.: PCT/CN2023/119655

§ 371 Date: Mar. 4, 2025

(30) Foreign Application Priority Data
Sep. 19, 2022 (WO) PCT/CN2022/119568

(51) Int. Cl. A61K 31/506; A61K 45/06; A61P 35/00; A61P 35/04

(52) U.S. Cl. CPC A61K 31/506; A61K 45/06; A61P 35/00; A61P 35/04

(57) ABSTRACT

Provided herein are a pharmaceutical composition containing a therapeutically effective amount of furmonertinib or a pharmaceutically acceptable salt thereof and optionally a pharmaceutically acceptable carrier, and use of furmonertinib or a pharmaceutically acceptable salt thereof, or said pharmaceutical composition in manufacture of a medicament for treating and/or preventing a disease mediated by PACC mutation. The pharmaceutical composition shows an excellent therapeutic effect on disease mediated by PACC mutation (for example, non-small cell lung cancer (NSCLC)) with little side effects and excellent safety.

### TECHNICAL FIELD

The present invention relates to a pharmaceutical composition comprising a therapeutically effective amount of furmonertinib or a pharmaceutically acceptable salt thereof and optionally a pharmaceutically acceptable carrier. The present invention also relates to use of furmonertinib or a pharmaceutically acceptable salt thereof, and the pharmaceutical composition in manufacture of a medicament for treating and/or preventing a disease mediated by PACC mutation (one subgroup of EGFR mutations, mutation on the interior surface of the ATP-binding pocket or C-terminal end of the αC-helix, which also referred as P-loop and αC-helix compressing mutation). The present invention also provides a method of treating and/or preventing a disease mediated by PACC mutation, comprising administering to a patient a therapeutically effective amount of furmonertinib or a pharmaceutically acceptable salt thereof.

### BACKGROUND TECHNOLOGY

Worldwide, lung cancer has always been a malignant tumor with the highest morbidity and mortality and serious harm to human health and life, and 1.76 million people died of lung cancer in 2018 all over the world. Non-small cell lung cancer (NSCLC) comprises approximately 80-85% of all lung cancers. Epidermal Growth Factor Receptor (EGFR) is a multifunctional glycoprotein widely distributed on the cell membrane of various tissues of the human body. The EGFR mutation is the most widely studied target in NSCLC.

Among EGFR mutations, common mutations include sensitive mutations (such as exon 19 deletion and exon 21 point mutation (L858R), comprising 85%-90% of all EGFR mutations), drug resistant mutations (such as exon 20 T790M mutation, exon 20 C797S mutation), etc. By using hierarchical clustering of in vitro selectivity over WT EGFR and mutational mapping of EGFR mutations, Robichaux etc. observed four distinct subgroups of EGFR mutations: classical-like mutations that were distant from the ATP-binding pocket, T790M-like mutations in the hydrophobic core, insertions in the loop at the C-terminal end of the «C-helix in exon 20, and mutations on the interior surface of the ATP-binding pocket or C-terminal end of the αC-helix, which were predicted to be P-loop and αC-helix compressing (PACC). PACC mutations include mutations spanning exons 18-21 including G719X, L747X, S768I, L792X, E709X, L718X, G724S, V769X and T854I, etc (Nature, volume 597, pages 732-737 (2021)).

Over the years, a large number of targeted drugs have been developed for EGFR mutation in NSCLC, such as the first generation of reversible tyrosinase inhibitor (TKI) Gefitinib and Erlotinib for EGFR sensitive mutation, the second generation of irreversible covalent binding inhibitor Afatinib, and the third generation of inhibitor Osimertinib for drug resistant mutation EGFR T790M, which have very good clinical effects.

However, no drug targeting EGFR PACC mutations has been approved in worldwide, so there is still a need to continue developing an effective, stable and safe small molecule drug as EGFR PACC mutation inhibitor.

N-{2-{[2-(dimethylamino)ethyl](methyl)amino}-6-(2,2,2-trifluoroethoxy)-5-{[4-(1-methyl-1H-indol-3-yl)pyrimidin-2-yl]amino}pyridin-3-yl}acrylamide (also referred to as “furmonertinib”) represented by the following formula (I) is described in patent CN105315259B, and the mesilate of the compound represented by the following formula (I) (also referred to as “furmonertinib mesilate”) is described in patent CN107163026B, and furmonertinib mesilate has been commercialized as a third-generation EGFR-TKI inhibitor, and is mainly used for treating a disease mediated by EGFR-sensitive mutation and T790M drug-resistant mutation. The phase I rising study of furmonertinib mesilate demonstrates that when furmonertinib mesilate is orally taken once per day at a dosage level of 20 mg-240 mg, the tolerance and the safety are good, adverse events of subjects are mild or moderate, dose-limiting toxicity does not occur, and dose-related toxic reaction does not occur; and in addition, the phase IIb clinical trial has demonstrated that the oral administration of 80 mg daily dose of furmonertinib mesilate shows a relatively good anti-tumor effect on patients with the EGFR T790M positive advanced non-small cell lung cancer, who has progressive disease after receiving prior systematic anti-tumor therapy, and can alleviate or stabilize the disease progression.

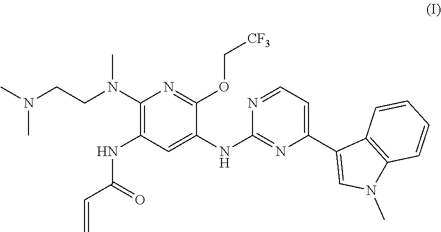 [SMILES: C=CC(=O)Nc1cc(Nc2nccc(-c3cn(C)c4ccccc34)n2)c(OCC(F)(F)F)nc1N(C)CCN(C)C]

### SUMMARY

The present invention provides, in some embodiments, use of furmonertinib or a pharmaceutically acceptable salt thereof.

In some embodiments, furmonertinib or a pharmaceutically acceptable salt thereof as an active compound can effectively inhibit PACC mutation, and thus, furmonertinib or a pharmaceutically acceptable salt thereof can be used for treating and/or preventing a disease mediated by PACC mutation.

Thus, in some embodiments, the present invention provides use of furmonertinib or a pharmaceutically acceptable salt thereof in manufacture of a medicament for treating and/or preventing a disease mediated by PACC mutation.

In some embodiments, the present invention provides use of furmonertinib or a pharmaceutically acceptable salt thereof in combination of at least one second therapeutic agent in manufacture of a medicament for treating and/or preventing a disease mediated by PACC mutation.

In some embodiments, furmonertinib or a pharmaceutically acceptable salt thereof is useful as an active compound at a certain dose, a disease mediated by PACC mutation, particularly non-small cell lung cancer, can be treated and/or prevented, and the treatment and/or prevention of the disease are/is accompanied by little side effects and is excellent in safety.

More specifically, the present invention provides a pharmaceutical composition comprising a therapeutically effective amount of furmonertinib, or a pharmaceutically acceptable salt thereof, and optionally a pharmaceutically acceptable carrier.

The present invention also provides use of the above-mentioned pharmaceutical composition of the present invention in manufacture of a medicament for treating and/or preventing a disease mediated by the PACC mutation.

The composition of the present invention can also be present in a formulation form of a tablet or a capsule. In each unit formulation, the content of furmonertinib or a pharmaceutically acceptable salt thereof is 10 mg-400 mg.

When the pharmaceutical composition of the present invention is used for treating and/or preventing a disease mediated by PACC mutation, the daily dose of furmonertinib or a pharmaceutically acceptable salt thereof may be 80 mg-400 mg. At this time, by adjusting the amount of the above-mentioned formulations (e.g. tablets or capsules), the daily dose of furmonertinib or a pharmaceutically acceptable salt thereof can be easily adjusted.

The present invention also provides a method of treating and/or preventing a disease mediated by PACC mutation, comprising administering to a patient a therapeutically effective amount of furmonertinib or a pharmaceutically acceptable salt thereof.

In the above treatment method of the present invention, it is desirable that the daily dose of furmonertinib or a pharmaceutically acceptable salt thereof is 80 mg-400 mg.

The present invention also provides a method of treating and/or preventing a disease comprising administering to a patient with positive PACC mutation a therapeutically effective amount of furmonertinib or a pharmaceutically acceptable salt thereof.

The present invention also provides a method of treating locally advanced or metastatic non-small cell lung cancer comprising administering to a patient in need thereof a therapeutically effective amount of furmonertinib or a pharmaceutically acceptable salt thereof.

The present invention also provides a method of treating locally advanced or metastatic non-small cell lung cancer comprising administering to a patient with confirmed positive PACC mutation a therapeutically effective amount of furmonertinib or a pharmaceutically acceptable salt thereof.

The present invention also provides a method of treating locally advanced or metastatic non-small cell lung cancer comprising administering to a patient harboring PACC mutation a therapeutically effective amount of furmonertinib or a pharmaceutically acceptable salt thereof.

The present invention also provides a method of treating locally advanced or metastatic non-small cell lung cancer comprising administering to a patient with confirmed positive PACC mutation who has received no prior systematic anti-tumor therapy a therapeutically effective amount of furmonertinib or a pharmaceutically acceptable salt thereof.

The present invention also provides a method of treating locally advanced or metastatic non-small cell lung cancer comprising administering to a patient with confirmed positive PACC mutation who has progressive disease after receiving prior systematic anti-tumor therapy a therapeutically effective amount of furmonertinib or a pharmaceutically acceptable salt thereof.

### Invention Effect

In the present invention, furmonertinib or a pharmaceutically acceptable salt thereof, or a pharmaceutical composition comprising furmonertinib or a pharmaceutically acceptable salt thereof and optionally a pharmaceutically acceptable carrier, exhibits excellent inhibitory activity against PACC mutation.

In addition, when furmonertinib or a pharmaceutically acceptable salt thereof, or a pharmaceutical composition comprising furmonertinib or a pharmaceutically acceptable salt thereof and optionally a pharmaceutically acceptable carrier of the present invention is used for treating and/or preventing a disease mediated by PACC mutation, the side effect is small and the safety is excellent.

The pharmaceutical composition of the present invention can be prepared into a formulation having an appropriate size and an appropriate content of active components by containing furmonertinib or a pharmaceutically acceptable salt thereof in a specific amount.

### DETAILED DESCRIPTION

Embodiments of the present invention will be described in more detail below with reference to specific embodiments, but those skilled in the art will appreciate that the specific embodiments described below are merely illustrative of the present invention and should not be construed as limiting the scope of the present invention. On the contrary, the present invention is intended to cover all alternatives, modifications and equivalents within the scope of the present invention as defined by the appended claims.

Unless otherwise specified, the embodiments of the present invention may be combined in any manner, and the conversions, modifications, and changes of the technical solutions obtained thereby are also included in the scope of the present invention.

Furmonertinib is a compound known in the prior art, described in particular in patent CN105315259B, with the chemical name: N-{2-{[2-(dimethylamino)ethyl](methyl)amino}-6-(2,2,2-trifluoroethoxy)-5-{[4-(1-methyl-1H-indol-3-yl)pyrimidin-2-yl]amino}pyridin-3-yl}acrylamide; the structural formula is the compound shown in the formula (I).

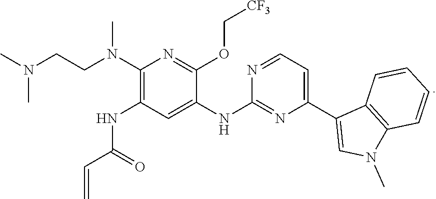 [SMILES: C=CC(=O)Nc1cc(Nc2nccc(-c3cn(C)c4ccccc34)n2)c(OCC(F)(F)F)nc1N(C)CCN(C)C]

In the present invention, the active component for the treatment of the disease is actually furmonertinib or a pharmaceutically acceptable salt thereof. Therefore, in the present invention, furmonertinib or a pharmaceutically acceptable salt thereof may be used alone or may be used by being contained in a composition, in which case the composition may optionally include a pharmaceutically acceptable carrier as desired.

In addition, in the present invention, furmonertinib or a pharmaceutically acceptable salt thereof can also be used in combination with at least one second therapeutic agent.

The present invention provides a pharmaceutical composition comprising a therapeutically effective amount of furmonertinib or a pharmaceutically acceptable salt thereof and optionally a pharmaceutically acceptable carrier.

“Pharmaceutically acceptable carrier” means one or more compatible solid or liquid fillers or gelatinous materials which are suitable for human use and should be of sufficient purity and sufficiently low toxicity. In the present invention, the carrier is also known as “adjuvant”. “Compatibility” means that each component in the composition can be admixed with the compounds of the present invention and with each other without significantly reducing the drug effect of the compounds. Some examples of pharmaceutically acceptable carriers include cellulose and derivatives thereof (such as sodium carboxymethyl cellulose, ethyl cellulose, methyl cellulose, hydroxypropylmethyl cellulose and derivatives thereof, cellulose acetate and derivatives thereof, cellulose acetate, etc.), gelatin, talc, solid lubricants (such as stearic acid, magnesium/calcium stearate, hydrogenated vegetable oil, sodium stearyl fumarate), calcium sulfate, vegetable oils (such as soybean oil, sesame oil, peanut oil, olive oil, etc.), polyols (such as propylene glycol, glycerol, mannitol, sorbitol, etc.), emulsifiers, wetting agents (such as sodium dodecyl sulfate), coloring agents, flavoring agents, stabilizers, antioxidants, preservatives, etc, but not limited thereto.

The pharmaceutical compositions of the present invention may be prepared by methods well known in the art, such as conventional mixing, dissolving, granulating, dragee-making, levigating, emulsifying, and lyophilizing processes.

The pharmaceutical compositions of the present invention may be prepared into the formation form of a tablet or a capsule, in the formulation, furmonertinib or a pharmaceutically acceptable salt thereof is mixed with at least one pharmaceutically acceptable carrier, in the present invention, the carrier is also known as “adjuvant”, said carrier may include but not limited to: (a) fillers or solubilizing agents, for example, microcrystalline cellulose, starch, lactose, sucrose, glucose, mannitol, colloidal silica, calcium hydrogen phosphate, calcium phosphate, calcium sulfate; (b) binders, for hydroxypropylmethylcellulose, hydroxypropylcellulose, example, methylcellulose, alginates, gelatin, polyvinylpyrrolidone, copovidone, sucrose and acacia, corn starch; (c) humectants, for example glycerin and the like; (d) disintegrants, for example, croscarmellose sodium, crospovidone, sodium carboxymethyl starch, colloidal silica, microcrystalline cellulose, potato starch or tapioca starch or corn starch, pregelatinized starch, alginic acid, certain complex silicates and sodium carbonate, ion exchange resins and the like; (e) absorption accelerators, for example, quaternary ammonium compounds, anionic or nonionic surfactants, cyclodextrins, and the like; (f) wetting agents such as cetyl alcohol and glycerol monostearate and the like; (g) adsorbents, for example, kaolin, colloidal silica, ion exchange resins, and the like; and (h) lubricants, for example, talc, calcium stearate, magnesium stearate, solid polyethylene glycols, sodium lauryl sulfate, sodium stearyl fumarate, hydrogenated vegetable oils, and the like, or mixtures thereof. The capsule and the tablet may also contain buffering agent. Tablets and capsules may be coated or microencapsulated with a coating or shell material such as an enteric coating or other materials known in the art.

The term “pharmaceutically acceptable salt” is a salt prepared from furmonertinib and a relatively non-toxic, pharmaceutically acceptable acid or base. Base addition salts may be obtained by contacting furmonertinib with a sufficient amount of a pharmaceutically acceptable base in pure solution or in a suitable inert solvent. Representative base addition salts include, for example, those salts formed with alkali metal, alkaline earth metal, quaternary ammonium cations such as sodium, lithium, potassium, calcium, magnesium, tetramethylquaternary ammonium, tetraethylquaternary ammonium, and the like; amine salts, including salts formed with ammonia (NH₃), primary, secondary or tertiary amines, such as methylamine salts, dimethylamine salts, trimethylamine salts, triethylamine salts, ethylamine salts, and the like. In addition, acid addition salts may be obtained by contacting furmonertinib with a sufficient amount of a pharmaceutically acceptable acid in pure solution or in a suitable inert solvent. The pharmaceutically acceptable acid salt comprises inorganic acid salts such as hydrochloride, sulfate, phosphate, and nitrate; and organic acid salts such as formate, acetate, propionate, methanesulfonate, benzylsulfonate, succinate, citrate, and tartrate. Reference can be specifically made to Berge et al., “Pharmaceutical Salts”, Journal of Pharmaceutical Science 66:1-19 (1977), or “Handbook of Pharmaceutical Salts: Properties, Selection, and Use” (P. Heinrich Stahl and Camille G. Wermuth, ed., Wiley-VCH, 2002).

In the present invention, “therapeutically effective amount” refers to a sufficient amount of drug or pharmacologically active agent that is non-toxic but yet achieves the desired effect. The effective amount will vary from person to person, depending on the age, weight and condition of the patient and also on the particular active substance, and an appropriate effective amount in individual cases may be determined by a person skilled in the art in the light of routine test.

In the present invention, “active component”, “active substance”, or “active agent” refers to a chemical entity that is effective in treating the disorder, disease, or condition of interest.

In the present invention, “patient”, “individual”, or “subject” includes humans, animals, vertebrates, mammals, rodents (e.g., guinea pigs, hamsters, rats, mice), murines (e.g., mice), canines (e.g., dogs), primates, anthropoids (e.g., monkeys or apes), monkeys (e.g., marmosets, baboons), apes (e.g., gorillas, chimpanzees, orangutans, gibbons). In some embodiments, “patient” is a human.

In the present invention, “treatment” refers to therapeutic treatment or palliative measures. When specific conditions are involved, treatment refers to: (1) relieving one or more biological manifestations of a disease or a disorder, (2) interfering with (a) one or more points in a biological cascade that causes or contributes to a disorder or (b) one or more biological manifestations of a disorder, (3) ameliorating one or more symptoms, effects, or side effects associated with a disorder, or one or more symptoms, effects, or side effects associated with a disorder or treatment thereof, or (4) slowing the progression of one or more biological manifestations of a disease or a disorder. “Treatment” may also refer to an increase in survival compared to expected survival without receiving the treatment.

In the present invention, “prevention” refers to a reduction in the risk of acquiring or developing a disease or a disorder.

In an embodiment of the present invention, the pharmaceutically acceptable salt of furmonertinib is a mesilate salt of furmonertinib, i.e., furmonertinib mesilate.

In an embodiment of the present invention, the pharmaceutical composition of the present invention is present in the formulation form of a tablet or a capsule.

In an embodiment of the present invention, the content of furmonertinib or a pharmaceutically acceptable salt thereof in per unit formulation (such as a tablet or a capsule) as described above is 10 mg-400 mg, preferably 20 mg-320 mg. As the specific content, for example, it can be 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the preferable specific content, it can be 20 mg, 40 mg, 80 mg, 160 mg, 240 mg or 320 mg, more preferably 40 mg or 80 mg, most preferably 40 mg.

In an embodiment of the present invention, in the pharmaceutical composition, the content of furmonertinib or a pharmaceutically acceptable salt thereof is 80 mg-400 mg, for example, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the preferable content, it can be 80 mg, 160 mg, 240 mg or 320 mg, more preferably 80 mg, 160 mg or 240 mg, most preferably 240 mg.

In an embodiment of the present invention, when the pharmaceutical composition is used for treating and/or preventing a disease mediated by PACC mutation, the composition is administered to a patient such that the dose of furmonertinib or a pharmaceutically acceptable salt thereof is 80 mg-400 mg. As the specific dose, for example, it can be 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the preferable dose, it can be 80 mg, 160 mg, 240 mg or 320 mg, more preferably 80 mg, 160 mg or 240 mg, most preferably 240 mg. In an embodiment of the present invention, the dose is a daily dose.

In other words, in the present invention, the content of furmonertinib or a pharmaceutically acceptable salt thereof in the pharmaceutical composition of the present invention refers to the total amount of furmonertinib or a pharmaceutically acceptable salt thereof in the pharmaceutical composition taken by a patient when said pharmaceutical composition is administered to the patient. For example, when a pharmaceutical composition is present in the formulation form of a tablet or a capsule, the content of furmonertinib or a pharmaceutically acceptable salt thereof in said pharmaceutical composition refers to the total amount of furmonertinib or a pharmaceutically acceptable salt thereof in all of the formulations when the formulations of tablets or capsules are administered.

It will be appreciated by those skilled in the art that when administered to a patient, the daily dose of furmonertinib or a pharmaceutically acceptable salt thereof is not less than the content of furmonertinib or a pharmaceutically acceptable salt thereof in per unit formulation. Those skilled in the art can calculate the total amount of the formulations that is necessary to be administered per day based on the daily dose of furmonertinib or a pharmaceutically acceptable salt thereof and the content of furmonertinib or a pharmaceutically acceptable salt thereof in each unit formulation. For example, when furmonertinib or a pharmaceutically acceptable salt thereof is contained in tablets and the content of furmonertinib or a pharmaceutically acceptable salt thereof in each unit formulation (each tablet) is 40 mg, and when the daily dose of furmonertinib or a pharmaceutically acceptable salt thereof is 240 mg, the total amount of the formulations (tablets) that is necessary to be administered per day is 6 tablets.

In an embodiment of the present invention, the pharmaceutical composition is administered 1, 2 or 3 times per day, preferably administered once per day, for the treatment and/or prevention of a disease mediated by PACC mutation.

In an embodiment of the present invention, the pharmaceutical composition may further comprise at least one second therapeutic agent. As the second therapeutic agent, it may be selected from chemotherapeutic drug, targeted antitumor drug, antibody drug and immunotherapeutic drug.

In an embodiment of the present invention, as said chemotherapeutic drug, the following can be exemplified: one or more of platinum drug (for example oxaliplatin, cisplatin, carboplatin, nedaplatin, dicycloplatin, lobaplatin, triplatinum tetranitrate, phenanthreneplatin, picoplatin, miriplatin, satraplatin), fluoropyrimidine derivative (for example gemcitabine, capecitabine, ancitabine, fluorouracil, tegadifur, doxifluridine, tegafur, carmofur, trifluridine, tegafur), camptothecins (for example camptothecin, hydroxycamptothecine, 9-amino camptothecin, 7-ethyl camptothecin, irinotecan, topotecan), taxels (for example paclitaxel, albumin-bound paclitaxel and docetaxel), vinblastines (vinorelbine, vinblastine, vincristine, vindesine, vinflunine), anthracenes (epirubicin, amycin, rubidomycin, pirarubicin, amrubicin, idarubicin, mitoxantrone, aclarubicin, valrubicin, zorubicin, pixantrone), antibiotics, podophyllums, antimetabolite antitumor drug, pemetrexed, carmustine, melphalan, etoposide, teniposide, mitomycin, iphosphamide, cyclophosphamide, azacitidine, methotrexate, bendamustine, liposome amycin, actinomycin D (dactinomycin), bleomycin, pingyangmycin, temozolomide, decarbazine, peplomycin, eribulin, plinabulin, sapacitabine, treosulfan, 153Sm-EDTMP, and encequidar.

In an embodiment of the present invention, said second therapeutic agent is one or more of platinum drug, and said platinum drug includes, but is not limited to Cisplatin, Carboplatin, Nedaplatin, oxaliplatin, triplatinum tetranitrate, phenanthreneplatin, picoplatin, satraplatin, miriplatin, Lobaplatin and the like.

In an embodiment of the present invention, said chemotherapeutic drug is selected from one or more of etoposide, irinotecan, cisplatin, carboplatin, lobaplatin, nedaplatin, topotecan, paclitaxel, docetaxel, temozolomide, vinorelbine, gemcitabine, cyclophosphamide, amycin, vincristine, bendamustine, pharmorubicin, methotrexate, amrubjcin, tegafur, gimeracil, oteracil, tegafur.

In an embodiment of the present invention, as the targeted antitumor drug, protein kinase inhibitors can be enumerated. Among them, the protein kinase inhibitors include but are not limited to tyrosine kinase inhibitors, serine and/or threonine kinase inhibitors, and poly ADP-ribose polymerase (PARP) inhibitors. The targets of the inhibitors include but are not limited to Fascin-1 protein, HDAC (histone deacetylase), Proteasome, CD38, SLAMF7 (CS1/CD319/CRACC), RANKL, EGFR (epidermal growth factor receptor), anaplastic lymphoma (ALK), METgene, ROS1gene, HER2gene, RETgene, BRAFgene, PI3K signal pathway, DDR2 (discoidin domain receptor 2) gene, FGFR1 (fibroblast growth factor receptor 1), NTRK1 (neurotrophic tyrosine kinase type 1 receptor) gene, and KRASgene. The targets of the targeted antitumor drug also include COX-2 (epoxidase-2), APE1 (apurinic-apyrimidinic endonuclease), VEGFR (vascular endothelial growth factor receptor), CXCR-4 (chemokine receptor-4), MMP (matrix metalloproteinase), IGF-1R (insulin-like growth factor receptor), Ezrin, PEDF (pigmented epithelial derived factor), AS, ES, OPG (bone protective factor), Src, IFN, ALCAM (activated leukocyte cell adhesion molecule), HSP, JIP1, GSK-3β (Glycogen Synthetic Kinase 3B), CyclinD1 (cell cycle regulator protein), CDK4 (cyclin-dependent kinase), TIMP1 (tissue metalloproteinase inhibitor), THBS3, PTHR1 (parathyroid hormone-related protein receptor 1), TEM7 (human tumor vascular endothelial marker 7), COPS3, and cathepsin K. The targeted antitumor drug that can be enumerated includes but is not limited to one or more of Imatinib, Sunitinib, Nilotinib, bosutinib, Saracatinib, Pazopanib, Trabectedin, Regorafenib, Cediranib, Bortezomib, Panobinostat, Carfilzomib, Ixazomib, apatinib, Erlotinib, Afatinib, Crizotinib, Ceritinib, Vemurafenib, Dabrafenib, Almonertinib, Osimertinib, Cabozantinib, Gefitinib, Dacomitinib, Olmutinib, Alectinib, Brigatinib, Lorlatinib, Trametinib, Larotrectinib, icotinib, Lapatinib, Vandetanib, Selumetinib, Sorafenib, Olmutinib, Savolitinib, Fruquintinib, Entrectinib, Dasatinib, Ensartinib, Lenvatinib, itacitinib, Pyrotinib, Binimetinib, Erdafitinib, Axitinib, Neratinib, Cobimetinib, Acalabrutinib, Famitinib, Masitinib, Ibrutinib, Anlotinib, rociletinib, nintedanib, Revlimid, LOXO-292, Vorolanib, bemcentinib, capmatinib, entrectinib, TAK-931, ALT-803, palbociclib, famitinib L-malate, LTT-462, BLU-667, ningetinib, tipifarnib, poziotinib, DS-1205c, capivasertib, SH-1028, Metformin, seliciclib, OSE-2101, APL-101, berzosertib, idelalisib, lerociclib, ceralasertib, PLB-1003, tomivosertib, SKLB-1028, D-0316, LY-3023414, allitinib, MRTX-849, AP-32788, AZD-4205, lifirafenib, vactosertib, mivebresib, napabucasin, sitravatinib, TAS-114, molibresib, CC-223, rivoceranib, CK-101, LXH-254, simotinib, GSK-3368715, TAS-0728, masitinib, tepotinib, HS-10296, AZD-4547, merestinib, olaptesed pegol, galunisertib, ASN-003, gedatolisib, defactinib, lazertinib, CKI-27, S-49076, BPI-9016M, RF-A-089, RMC-4630, AZD-3759, antroquinonol, SAF-189s, AT-101, TTI-101, naputinib, LNP-3794, HH-SCC-244, ASK-120067, CT-707, epitinib succinate, tesevatinib, SPH-1188-11, BPI-15000, copanlisib, niraparib, olaparib, veliparib, talazoparib tosylate, DV-281, Siremadlin, Telaglenastat, MP-0250, GLG-801, ABTL-0812, bortezomib, tucidinostat, vorinostat, resminostat, epacadostat, tazemetostat, entinostat, mocetinostat, quisinostat, LCL-161, and KML-001. In some embodiments, the targeted antitumor drug is one or more of Sorafenib, Erlotinib, Afatinib, Crizotinib, Ceritinib, Vemurafenib, Dabrafenib, Cabozantinib, Gefitinib, Dacomtinib, Osimertinib, Alectinib, Brigatinib, Lorlatinib, Trametinib, Larotrectinib, Icotinib, Lapatinib, Vandetanib, Selumetinib, Olmutinib, Savolitinib, Fruquintinib, Entrectinib, Dasatinib, Ensartinib, Lenvatinib, Itacitinib, Pyrotinib, Binimetinib, Erdafitinib, Axitinib, Niratinib, Cobimetinib, Acalabrutinib, Famitinib, Masitinib, Ibrutinib, Anlotinib, Nintedanib.

In an embodiment of the present invention, the second therapeutic agent is an antibody drug. Among others, the targets aimed by the antibody drug include but are not limited to any one or more of PD-1, PD-L1, cytotoxic T-lymphocyte antigen 4 (CTLA-4), platelet-derived growth factor receptor α (PDGFR-α), vascular endothelial growth factor (VEGF), human epidermal growth factor receptor-2 (HER2), epidermal growth factor receptor (EGFR), ganglioside GD2, B-cell surface protein CD20, B-cell surface protein CD52, B-cell surface protein CD38, B-cell surface protein CD319, B-cell surface protein CD30, and B-cell surface protein CD19/CD3.

In an embodiment of the present invention, the antibody drug is an inhibitor for the interaction between the PD-1 receptor and its ligand PD-L1; in an embodiment of the present invention, the antibody drug is cytotoxic T-lymphocyte antigen 4 inhibitor. In an embodiment of the present invention, the antibody drug is platelet-derived growth factor receptor α (PDGFR-α) inhibitor.

In an embodiment of the present invention, the inhibitor for the interaction between the PD-1 receptor and its ligand PD-L1 is an antibody or its antigen-binding portion that binds to the programmed death receptor 1 (PD-1) and/or inhibits the activity of PD-1, or an antibody or its antigen-binding portion that binds to the programmed death ligand 1 (PD-L1) and/or inhibits the activity of PD-L1, for example, an anti-PD-1 antibody or an anti-PD-L1 antibody. In an embodiment of the present invention, the antibody or its antigen-binding portion is (a) an anti-PD-1 monoclonal antibody or its antigen-binding fragment, which specifically binds to human PD-1 and blocks the binding between human PD-L1 and human PD-1; or (b) an anti-PD-L1 monoclonal antibody or its antigen-binding fragment, which specifically binds to human PD-L1 and blocks the binding between human PD-L1 and human PD-1.

In an embodiment of the present invention, the anti-PD-1 or PD-L1 antibody is an anti-PD-1 or PD-L1 monoclonal antibody.

In an embodiment of the present invention, the anti-PD-1 or PD-L1 antibody is a human antibody or a murine antibody.

In an embodiment of the present invention, the anti-PD-1 antibody can be selected from any one or more of Nivolumab, Pembrolizumab, Durvalumab, Toripalimab (JS-001), Sintilimab (IBI308), Camrelizumab, Tislelizumab (BGB-A317), Geptanolimab (GB226), Lizumab (LZM009), HLX-10, BAT-1306, AK103 (HX008), AK104 (Akesobio), CS1003, SCT-I10A, F520, SG001, and GLS-010.

In an embodiment of the present invention, the anti-PD-L1 antibody can be selected from any one or more of Atezolizumab, Avelumab, Durvalumab, KL-A167, BGB-333, SHR-1316, JS003, STI-A1014 (ZKAB0011), KN035, MSB2311, HLX-20, and CS-1001.

In an embodiment of the present invention, the anti-PD-1 antibody is Toripalimab.

In an embodiment of the present invention, the anti-PD-1 antibody is Pembrolizumab.

In an embodiment of the present invention, the cytotoxic T-lymphocyte antigen 4 (CTLA-4) inhibitor is an anti-CTLA-4 antibody, in an embodiment of the present invention, the anti-CTLA-4 antibody is an anti-CTLA-4 monoclonal antibody.

In an embodiment of the present invention, the anti-CTLA-4 antibody can be selected from any one or more of Ipilimumab, Tremelimumab, AGEN-1884, BMS-986249, BMS-986218, AK-104, and IBI310.

In an embodiment of the present invention, the anti-CTLA-4 antibody is Ipilimumab.

In an embodiment of the present invention, the platelet-derived growth factor receptor α (PDGFR-α) inhibitor is an anti-PDGFR α antibody. In an embodiment of the present invention, the anti-PDGFRα antibody is an anti-PDGFRα monoclonal antibody.

In an embodiment of the present invention, the anti-PDGFRα antibody is Olaratumab.

In an embodiment of the present invention, the antibody drug can also include, but are not limited to any one or more of Bevacizumab, Ramucirumab, Pertuzumab, Trastuzmab, Cotuximab, Nimotuzumab, Panitumumab, Necitumumab, Dinutuximab, Rituximab, Ibritumomab, Ofatumumab, Obinutuzumab, Alemtuzumab, Daratumumab, Gemtuzumab, Elotuzumab, Brentuximab, Inotuzumab Ozogamicin, Blinatumomab.

In an embodiment of the present invention, as immunotherapeutic drug, the following can be enumerated: one or more interferon (interferon α, interferon α-1b, interferon α-2b), interleukin, temsirolimus, everolimus, ridaforolimus, and temsirolimus.

In an embodiment of the present invention, when a second therapeutic agent is used, the amount of the second therapeutic agent can be adjusted as desired by those skilled in the art.

The present invention also provides use of furmonertinib or a pharmaceutically acceptable salt thereof in manufacture of a medicament for treating and/or preventing a disease mediated by PACC mutation.

The present invention also provides use of furmonertinib or a pharmaceutically acceptable salt thereof in combination of at least one second therapeutic agent in manufacture of a medicament for treating and/or preventing a disease mediated by PACC mutation.

The present invention also provides use of the above-mentioned pharmaceutical composition in manufacture of a medicament for treating and/or preventing a disease mediated by PACC mutation.

In an embodiment of the present invention, in the above use of the present invention, the pharmaceutically acceptable salt of furmonertinib is a mesilate salt of furmonertinib, i.e., furmonertinib mesilate.

In an embodiment of the present invention, in the above use of the present invention, the pharmaceutical composition of the present invention is present in the formulation form of a tablet or a capsule.

In an embodiment of the present invention, in the above use of the present invention, the content of said furmonertinib or a pharmaceutically acceptable salt thereof in per unit formulation (such as a tablet or a capsule) as described above is 10 mg-400 mg, preferably 20 mg-320 mg. As the specific content, it can be for example 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. Preferably, as the specific content, it can be 20 mg, 40 mg, 80 mg, 160 mg, 240 mg or 320 mg, more preferably 40 mg or 80 mg, most preferably 40 mg.

In an embodiment of the present invention, in the above use of the present invention, in said pharmaceutical composition, the content of furmonertinib or a pharmaceutically acceptable salt thereof is 80 mg-400 mg, for example 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the preferable content, it can be 80 mg, 160 mg, 240 mg or 320 mg, more preferably 80 mg, 160 mg or 240 mg, most preferably 240 mg.

In an embodiment of the present invention, in the above use of the present invention, the pharmaceutical composition is administered to a patient such that the dose of furmonertinib or a pharmaceutically acceptable salt thereof is 80 mg-400 mg. As the specific dose, it can be for example 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the preferable dose, it can be 80 mg, 160 mg, 240 mg or 320 mg, more preferably 80 mg, 160 mg or 240 mg, most preferably 240 mg. In an embodiment of the present invention, the dose is a daily dose.

In other words, in the above use of the present invention, the content of furmonertinib or a pharmaceutically acceptable salt thereof in the pharmaceutical composition refers to the total amount of furmonertinib or a pharmaceutically acceptable salt thereof in the pharmaceutical composition taken by a patient when said pharmaceutical composition is administered to the patient. For example, when a pharmaceutical composition is present in the formulation form of a tablet or a capsule, the content of furmonertinib or a pharmaceutically acceptable salt thereof in said pharmaceutical composition refers to the total amount of furmonertinib or a pharmaceutically acceptable salt thereof in all of the formulations when the formulations of tablets or capsules are administered.

It will be appreciated by those skilled in the art that in the use described herein, when administered to a patient, the daily dose of furmonertinib or a pharmaceutically acceptable salt thereof is not less than the content of furmonertinib or a pharmaceutically acceptable salt thereof in per unit formulation. Those skilled in the art can calculate the total amount of the formulations that is necessary to be administered per day based on the daily dose of furmonertinib or a pharmaceutically acceptable salt thereof and the content of furmonertinib or a pharmaceutically acceptable salt thereof in each unit formulation. For example, when furmonertinib or a pharmaceutically acceptable salt thereof is contained in tablets and the content of furmonertinib or a pharmaceutically acceptable salt thereof in each unit formulation (each tablet) is 40 mg, and when the daily dose of furmonertinib or a pharmaceutically acceptable salt thereof is 240 mg, the total amount of the formulations (tablets) that is necessary to be administered per day is 6 tablets.

In an embodiment of the present invention, the disease mediated by PACC mutation is cancer, for example lung cancer, and further can be non-small cell lung cancer (NSCLC).

In an embodiment of the present invention, the disease mediated by PACC mutation is locally advanced non-small cell lung cancer or metastatic non-small cell lung cancer.

In an embodiment of the present invention, the disease mediated by PACC mutation is a treatment-naive non-small cell lung cancer or a previously-treated non-small cell lung cancer.

As used herein, the term “treatment-naive” refers to a condition where before receiving the treatment with furmonertinib or a pharmaceutically acceptable salt thereof of the present invention, the treatment with another therapeutic agent (including but not limited to chemotherapeutic drug, targeted antitumor drug, antibody drug or immunotherapeutic drug) has not been used, or a condition where no systematic anti-tumor therapy has been taken. The term “previously-treated” refers to a condition where before receiving the treatment with furmonertinib or a pharmaceutically acceptable salt thereof of the present invention, the treatment with another therapeutic agent (including but not limited to chemotherapeutic drug, targeted antitumor drug, antibody drug or immunotherapeutic drug) has been used, or a condition where a systematic anti-tumor therapy has been taken, but afterwards the disease has progressed. In the case of “previously-treated”, the patient may have developed the resistance to other therapeutic agents, or may not develop the drug resistance.

In an embodiment of the present invention, the PACC mutation is at least one selected from a group consisting of EGFR-G719S mutation, EGFR-S768I mutation, EGFR-G724S mutation, EGFR-L718Q mutation, EGFR-Del19/G724S mutation, EGFR-E709H mutation, EGFR-L747S mutation, EGFR-E709V mutation, EGFR-L747V mutation, EGFR-E709-710>D mutation, EGFR-E709A mutation, EGFR-Del19/C797S mutation, EGFR-L858R/C797S mutation, EGFR-L747S/G719A mutation, EGFR-L858R-L718V mutation, EGFR-L858R-L718Q mutation, EGFR-E709K-G719A mutation and EGFR-L861Q mutation.

In an embodiment of the present invention, the PACC mutation is at least one selected from a group consisting of EGFR-G719S mutation, EGFR-S768I mutation, EGFR-G724S mutation, EGFR-L718Q mutation, EGFR-Del19/G724S mutation, EGFR-E709H mutation, EGFR-L747S mutation, EGFR-E709V mutation, EGFR-L747V mutation, EGFR-E709-710>D mutation, EGFR-E709A mutation, EGFR-Del19/C797S mutation, EGFR-L858R/C797S mutation, EGFR-L747S/G719A mutation, EGFR-L858R-L718V mutation, EGFR-L858R-L718Q mutation, EGFR-E709K-G719A mutation, EGFR-G719A mutation, EGFR-V769L mutation and EGFR-L861Q mutation.

In an embodiment of the present invention, the PACC mutation is at least one selected from a group consisting of EGFR-G719S mutation, EGFR-S768I mutation, EGFR-G724S mutation, EGFR-Del19/G724S mutation, EGFR-E709H mutation, EGFR-L747S mutation, EGFR-E709V mutation, EGFR-L747V mutation, EGFR-E709-710>D mutation, EGFR-E709A mutation, EGFR-L747S/G719A mutation, EGFR-L858R-L718V mutation, EGFR-E709K-G719A mutation and EGFR-L861Q mutation.

In an embodiment of the present invention, the PACC mutation is at least one selected from a group consisting of EGFR-G719S mutation, EGFR-S768I mutation, EGFR-G724S mutation, EGFR-Del19/G724S mutation, EGFR-E709H mutation, EGFR-L747S mutation, EGFR-E709V mutation, EGFR-L747V mutation, EGFR-E709-710>D mutation, EGFR-E709A mutation, EGFR-L747S/G719A mutation, EGFR-L858R-L718V mutation, EGFR-E709K-G719A mutation, EGFR-G719A mutation, EGFR-V769L mutation and EGFR-L861Q mutation.

In an embodiment of the present invention, in the use described herein, the pharmaceutical composition can further comprise at least one second therapeutic agent.

In the use described herein, the second therapeutic agent can be selected from chemotherapeutic drug, targeted antitumor drug, antibody drug and immunotherapeutic drug.

In the use described herein, the second therapeutic agent is the above-mentioned second therapeutic agent of the present invention.

The present invention provides a method of treating and/or preventing a disease mediated by PACC mutation, comprising administering to a patient a therapeutically effective amount of furmonertinib or a pharmaceutically acceptable salt thereof.

The present invention provides a method of treating and/or preventing a disease comprising administering to a patient with positive PACC mutation a therapeutically effective amount of furmonertinib or a pharmaceutically acceptable salt thereof.

The present invention provides a method of treating locally advanced or metastatic non-small cell lung cancer, comprising administering to a patient in need thereof a therapeutically effective amount of furmonertinib or a pharmaceutically acceptable salt thereof.

The present invention provides a method of treating locally advanced or metastatic non-small cell lung cancer, comprising administering to a patient with confirmed positive PACC mutation a therapeutically effective amount of furmonertinib or a pharmaceutically acceptable salt thereof.

The present invention provides a method of treating locally advanced or metastatic non-small cell lung cancer, comprising administering to a patient harboring PACC mutation a therapeutically effective amount of furmonertinib or a pharmaceutically acceptable salt thereof.

The present invention provides a method of treating locally advanced or metastatic non-small cell lung cancer, comprising administering to a patient with confirmed positive PACC mutation who has received no prior systematic anti-tumor therapy a therapeutically effective amount of furmonertinib or a pharmaceutically acceptable salt thereof.

The present invention provides a method of treating locally advanced or metastatic non-small cell lung cancer, comprising administering to a patient with confirmed positive PACC mutation who has progressive disease after receiving prior systematic anti-tumor therapy a therapeutically effective amount of furmonertinib or a pharmaceutically acceptable salt thereof.

In the above treatment method of the present invention, the furmonertinib or a pharmaceutically acceptable salt thereof is administrated at a dose of 80 mg-400 mg. As the specific dose, it can be for example 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. As the preferable dose, it can be 80 mg, 160 mg, 240 mg or 320 mg, more preferably 80 mg, 160 mg or 240 mg, most preferably 240 mg. In an embodiment of the present invention, the dose is a daily dose.

In the above treatment method of the present invention, the frequency at which furmonertinib or a pharmaceutically acceptable salt thereof is administered to a patient is 1 time per day (qd), 2 times per day (bid), or 3 times per day (tid), preferably 1 time per day.

In the above treatment method of the present invention, furmonertinib or a pharmaceutically acceptable salt thereof is administered to a patient under fasted state, preferably under fasted state in the morning.

In the above treatment method of the present invention, furmonertinib or a pharmaceutically acceptable salt thereof is orally administered to a patient.

In the above treatment method of the present invention, furmonertinib mesilate is administered to a patient.

In the above treatment method of the present invention, furmonertinib or a pharmaceutically acceptable salt thereof is administered in the formulation form of a tablet or a capsule.

In the above treatment method of the present invention, furmonertinib or a pharmaceutically acceptable salt thereof is administered to a patient in the form of each unit formulation. By adjusting the amount of unit formulation, the daily dose of furmonertinib or a pharmaceutically acceptable salt thereof is in the above range.

In the above treatment method of the present invention, in each unit formulation (such as a tablet or a capsule), the content of said furmonertinib or a pharmaceutically acceptable salt thereof is 10 mg-400 mg, preferably 20 mg-320 mg. As the specific content, it can be for example 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg. Preferably as the specific content, it can be 20 mg, 40 mg, 80 mg, 160 mg, 240 mg or 320 mg, more preferably 40 mg or 80 mg, most preferably 40 mg.

It will be appreciated by those skilled in the art that when administered to a patient, the daily dose of furmonertinib or a pharmaceutically acceptable salt thereof is not less than the content of furmonertinib or a pharmaceutically acceptable salt thereof in per unit formulation. Those skilled in the art can calculate the total amount of the formulations that is necessary to be administered per day based on the daily dose of furmonertinib or a pharmaceutically acceptable salt thereof and the content of furmonertinib or a pharmaceutically acceptable salt thereof in each unit formulation. For example, when furmonertinib or a pharmaceutically acceptable salt thereof is contained in tablets and the content of furmonertinib or a pharmaceutically acceptable salt thereof in each unit formulation (each tablet) is 40 mg, and when the daily dose of furmonertinib or a pharmaceutically acceptable salt thereof is 240 mg, the total amount of the formulations (tablets) that is necessary to be administered per day is 6 tablets.

In the above treatment method of the present invention, at least one second therapeutic agent can be further administered to a patient. In the above treatment method of the present invention, as the second therapeutic agent, it can be selected from chemotherapeutic drug, targeted antitumor drug, antibody drug and immunotherapeutic drug.

In the above treatment method of the present invention, the second therapeutic agent is the above-mentioned second therapeutic agent of the present invention.

In the above treatment method of the present invention, the disease is cancer, for example lung cancer, and further can be non-small cell lung cancer (NSCLC).

In the above treatment method of the present invention, furmonertinib or a pharmaceutically acceptable salt thereof is administered to a patient before or after surgical resection of tumor.

In the above treatment method of the present invention, the disease is locally advanced non-small cell lung cancer or metastatic non-small cell lung cancer.

In the above treatment method of the present invention, the disease is a treatment-naive non-small cell lung cancer or a previously-treated non-small cell lung cancer.

In the above treatment method of the present invention, the PACC mutation is at least one selected from a group consisting of EGFR-G719S mutation, EGFR-S768I mutation, EGFR-G724S mutation, EGFR-L718Q mutation, EGFR-Del19/G724S mutation, EGFR-E709H mutation, EGFR-L747S mutation, EGFR-E709V mutation, EGFR-L747V mutation, EGFR-E709-710>D mutation, EGFR-E709A mutation, EGFR-Del19/C797S mutation, EGFR-L858R/C797S mutation, EGFR-L747S/G719A mutation, EGFR-L858R-L718V mutation, EGFR-L858R-L718Q mutation, EGFR-E709K-G719A mutation and EGFR-L861Q mutation.

In the above treatment method of the present invention, the PACC mutation is at least one selected from a group consisting of EGFR-G719S mutation, EGFR-S768I mutation, EGFR-G724S mutation, EGFR-L718Q mutation, EGFR-Del19/G724S mutation, EGFR-E709H mutation, EGFR-L747S mutation, EGFR-E709V mutation, EGFR-L747V mutation, EGFR-E709-710>D mutation, EGFR-E709A mutation, EGFR-Del19/C797S mutation, EGFR-L858R/C797S mutation, EGFR-L747S/G719A mutation, EGFR-L858R-L718V mutation, EGFR-L858R-L718Q mutation, EGFR-E709K-G719A mutation, EGFR-G719A mutation, EGFR-V769L mutation and EGFR-L861Q mutation.

In an embodiment of the present invention, the PACC mutation is at least one selected from a group consisting of EGFR-G719S mutation, EGFR-S768I mutation, EGFR-G724S mutation, EGFR-Del19/G724S mutation, EGFR-E709H mutation, EGFR-L747S mutation, EGFR-E709V mutation, EGFR-L747V mutation, EGFR-E709-710>D mutation, EGFR-E709A mutation, EGFR-L747S/G719A mutation, EGFR-L858R-L718V mutation, EGFR-E709K-G719A mutation and EGFR-L861Q mutation.

In an embodiment of the present invention, the PACC mutation is at least one selected from a group consisting of EGFR-G719S mutation, EGFR-S768I mutation, EGFR-G724S mutation, EGFR-Del19/G724S mutation, EGFR-E709H mutation, EGFR-L747S mutation, EGFR-E709V mutation, EGFR-L747V mutation, EGFR-E709-710>D mutation, EGFR-E709A mutation, EGFR-L747S/G719A mutation, EGFR-L858R-L718V mutation, EGFR-E709K-G719A mutation, EGFR-G719A mutation, EGFR-V769L mutation and EGFR-L861Q mutation.

In the above treatment method of the present invention, the patient is a human patient.

In the above treatment method of the present invention, the patient is between age 18 and 75.

In the above treatment method of the present invention, the patient has histologically or cytopathologically confirmed primary non-small cell lung cancer (NSCLC) with predominant non-squamous cell histology prior to the start of treatment with furmonertinib or a pharmaceutically acceptable salt thereof.

In the above treatment method of the present invention, the patient has radiological disease progression following the last anti-tumor therapy prior to the start of treatment with furmonertinib or a pharmaceutically acceptable salt thereof.

In the above treatment method of the present invention, the patient has documented positive PACC mutation by laboratory test prior to the start of treatment with furmonertinib or a pharmaceutically acceptable salt thereof.

In the above treatment method of the present invention, the patient has locally advanced non-small cell lung cancer or metastatic non-small cell lung cancer and is confirmed to have radiological or pathological disease progression during or after the last systematic anti-tumor therapy prior to the start of treatment with furmonertinib or a pharmaceutically acceptable salt thereof.

In the above treatment method of the present invention, the patient has locally advanced non-small cell lung cancer or metastatic non-small cell lung cancer and has received no prior systematic anti-tumor therapy prior to the start of treatment with furmonertinib or a pharmaceutically acceptable salt thereof.

In the above treatment method of the present invention, the patient has at least one measurable lesion prior to the start of treatment with furmonertinib or a pharmaceutically acceptable salt thereof.

In the above treatment method of the present invention, the patient has adequate organ function as shown by laboratory test prior to the start of treatment with furmonertinib or a pharmaceutically acceptable salt thereof.

In the above treatment method of the present invention, the patient is subjected to an ECOG PS (Eastern Cooperative Oncology Group performance status) score test, such as an ECOG PS score of 0-1, prior to the start of treatment with furmonertinib or a pharmaceutically acceptable salt thereof.

The above treatment method of the present invention has an acceptable safety profile.

The above treatment method of the present invention can provide the therapeutic efficacy of partial response (PR).

The above treatment method of the present invention can provide the therapeutic efficacy of stable disease (SD).

The above treatment method of the present invention can provide tumor shrinkage in target lesions.

In the above treatment method of the present invention, tumor shrinkage in target lesions is provided, as evaluated by tumor radiological examination, such as computed tomography (CT) and/or magnetic resonance imaging (MRI).

### BRIEF DESCRIPTION OF THE DRAWINGS

FIG. 1: The curves of tumor volume change in Test Example 2.

FIG. 2: The curves of body weight change in Test Example 2.

### EXAMPLES

### I. Preparation Examples

### Preparation of Furmonertinib Mesilate, 40 mg, Standard Tablet

Formula: furmonertinib mesilate 46.76 mg, microcrystalline cellulose 44.73 mg, lactose 68.2 mg, croscarmellose sodium 13 mg, polyethylene glycol 4000 17.8 mg, colloidal silica 10.9 mg, sodium stearyl fumarate 2.7 mg, sodium chloride 8.67 mg, and 40 mg furmonertinib contained therein.

Process: sieving adjuvants and the active pharmaceutical ingredient for pretreatment and mixing uniformly, adding an appropriate amount of Polyethylene Glycol 4000 for wet granulation, sieving out for wet granulation, drying the wet granules, sieving out for granulation, adding colloidal silica and sodium stearyl fumarate and mixing uniformly, and then tabletting to obtain tablets.

### II. Activity Examples

### Test Example 1: Evaluation of Anti-Proliferation Effect of Furmonertinib Mesilate on 18 Cell Lines

The objective of the test example 1 was to evaluate the anti-proliferation effect of Furmonertinib mesilate on 18 cell lines (Ba/F3 EGFR-G719S, Ba/F3 EGFR-S768I, Ba/F3 EGFR-G724S, Ba/F3 EGFR-L718Q, Ba/F3 EGFR-Del19/G724S, Ba/F3 EGFR-E709H, Ba/F3 EGFR-L747S, Ba/F3 EGFR-E709V, Ba/F3 EGFR-L747V, Ba/F3 EGFR-E709-710>D, Ba/F3 EGFR-E709A, Ba/F3 EGFR-Del19/C797S, Ba/F3 EGFR-L858R/C797S, Ba/F3 EGFR-L747S/G719A, Ba/F3-EGFR-L858R-L718V, Ba/F3-EGFR-L858R-L718Q, Ba/F3-EGFR-E709K-G719A, and Ba/F3 EGFR-L861Q) using standard CellTiter-Glo® Luminescent Cell Viability Assay. Further, AZD9291 was used as control compound.

Cell culture: Ba/F3 EGFR-G719S, Ba/F3 EGFR-S768I, Ba/F3 EGFR-G724S, Ba/F3 EGFR-L718Q, Ba/F3 EGFR-Del19/G724S, Ba/F3 EGFR-E709H, Ba/F3 EGFR-L747S, Ba/F3 EGFR-E709V, Ba/F3 EGFR-L747V, Ba/F3 EGFR-E709-710>D, Ba/F3 EGFR-E709A, Ba/F3 EGFR-Del19/C797S, Ba/F3 EGFR-L858R/C797S, Ba/F3 EGFR-L747S/G719A, Ba/F3-EGFR-L858R-L718V, Ba/F3-EGFR-L858R-L718Q, Ba/F3-EGFR-E709K-G719A, and Ba/F3 EGFR-L861Q engineered cells were obtained from Kyinno, and maintained at 37° C. with 5% CO₂, in the RPMI1640 media supplemented with 10% fetal bovine serum.

The cells during the logarithmic growth period were harvested and counted by using blood counting chamber. The cell viability was over 90% which was determined by trypan blue assay. The cell concentration was adjusted with culture medium. 90 μl cell suspensions was added to two 96-well plates to obtain the final cell density of 3000 cells per well. The plates were incubated in humidified incubator at 37° C. with 5% CO₂.

The solution of tested compounds (Furmonertinib mesilate and AZD9291) was diluted 1000-fold with DMSO. Then, said DMSO solution was diluted 100-fold with PBS (Phosphate Buffered Saline) to obtain 10× working solution. And 10× reference control solution was prepared by Phosphate Buffered Saline (PBS). 10 μl working solution (10×) was dispensed to each well (triplicate for each concentration). (The top concentration was 3160 nM, 3.16-fold dilution and 10 different concentrations. The final concentration of DMSO in culture medium was 0.1% [v/v]). The plate was cultured for 3 days.

On Day 3, CellTiter-Glo® reagent (a luciferase ATP bioluminescence detection reagent) was thawed, and then the cell plate was equilibrated at room temperature for approximately 30 minutes. 100 μl CellTiter-Glo® Reagent which was equal to the volume of cell culture medium present in each well was added. The obtained content was mixed for 5 minutes on an orbital shaker to induce cell lysis. The cell plate was incubated at room temperature for 20 minutes to stabilize luminescent signal. The luminescence was recorded by using Multi-mode Microplate Reader.

$$Cell Viability ( \% ) = \frac{{Lum}_{test aricles} - {Lum}_{blank control}}{{Lum}_{vehicle control} - {Lum}_{blank control}} \times 100 \%$$

blank control: medium control;
vehicle control: culture medium with 0.1% DMSO

The software of GraphPad Prism 7.0 was used to calculate IC₅₀, the IC₅₀ values were shown in Table 1. The graphical curves were fitted using a nonlinear regression model with a sigmoidal dose response.

TABLE 1

| | | IC₅₀ (nM) | |
|---|---|---|---|
| No. | Cell line | Furmonertinib mesilate | AZD9291 |
| 1 | Ba/F3 EGFR-G719S | 12.14 | 55.37 |
| 2 | Ba/F3 EGFR-S768I | 21.59 | 85.51 |
| 3 | Ba/F3 EGFR-G724S | 21.31 | 72.99 |
| 4 | Ba/F3 EGFR-L718Q | 500.78 | >1000 |
| 5 | Ba/F3 EGFR-Del19/G724S | 3.84 | 16.27 |
| 6 | Ba/F3 EGFR-E709H | 31.31 | 123.59 |
| 7 | Ba/F3 EGFR-L747S | 14.97 | 37.76 |
| 8 | Ba/F3 EGFR-E709V | 14.28 | 47.64 |
| 9 | Ba/F3 EGFR-L747V | 31.62 | 53.71 |
| 10 | Ba/F3 EGFR-E709-710 > D | 89.60 | 197.39 |
| 11 | Ba/F3 EGFR-E709A | 17.89 | 64.25 |
| 12 | Ba/F3 EGFR-Del19/C797S | 1220.84 | 1975.58 |
| 13 | Ba/F3 EGFR-L858R/C797S | 2208.50 | 2188.52 |
| 14 | Ba/F3 EGFR-L747S/G719A | 16.51 | 92.10 |
| 15 | Ba/F3-EGFR-L858R-L718V | 23.86 | 172.00 |
| 16 | Ba/F3-EGFR-L858R-L718Q | 289.41 | 942.31 |
| 17 | Ba/F3-EGFR-E709K-G719A | 38.95 | 163.67 |
| 18 | Ba/F3 EGFR-L861Q | 3.80 | 24.56 |

The result showed that furmonertinib mesilate had good anti-proliferation effect on Ba/F3 EGFR-G719S, Ba/F3 EGFR-S768I, Ba/F3 EGFR-G724S, Ba/F3 EGFR-L718Q, Ba/F3 EGFR-Del19/G724S, Ba/F3 EGFR-E709H, Ba/F3 EGFR-L747S, Ba/F3 EGFR-E709V, Ba/F3 EGFR-L747V, Ba/F3 EGFR-E709-710>D, Ba/F3 EGFR-E709A, Ba/F3 EGFR-Del19/C797S, Ba/F3 EGFR-L858R/C797S, Ba/F3 EGFR-L747S/G719A, Ba/F3-EGFR-L858R-L718V, Ba/F3-EGFR-L858R-L718Q, Ba/F3-EGFR-E709K-G719A, and Ba/F3 EGFR-L861Q cells.

### Test Example 2: Testing the Anti-Tumor Effect of Furmonertinib Mesilate in Ba/F3 EGFR-G724S (KC-1385) Cell Xenograft Model of Female Immune Deficiency Mice

This study was used for evaluate the anti-tumor efficacy of furmonertinib mesilate in the female (B-NDG) immune deficiency mice bearing tumors of Ba/F3 EGFR-G724S engineered cells.

Experimental animals: B-NDG mice, female, 6-8 weeks, and weighing 18-20 g.

Animal modeling and random grouping: Ba/F3 EGFR-G724S cells were cultivated, cells were collected, and re-suspended and counted in serum-free medium, the re-suspended cells with the addition of a matrigel at 1:1 were subcutaneous inoculated at 1×10⁶ cells/0.1 mL in B-NDG mice. When the average tumor volume was about 80-120 mm³, the animals were randomly grouped into four experiment groups according to the tumor size. Each group contained 12 mice. The grouping day was defined as Day 0, i.e., D0.

The experimental scheme: B-NDG mice were subcutaneously inoculated with Ba/F3 EGFR-G724S cells to establish a cell-line-derived xenograft model. The experiment was divided into 15 mg/kg group of furmonertinib mesilate, 30 mg/kg group of furmonertinib mesilate, 50 mg/kg group of furmonertinib mesilate and vehicle group, wherein each group contained 12 animals, orally administered with the administration volume of 10 uL/g, and the vehicle group was administered with the same amount of vehicle, the administration was carried out once per day and lasted for two weeks. During the whole experiment, the body weight and the tumor sizes of the mice were measured twice each week, and whether or not the presence of toxic reactions was observed.

Tumor Volume (TV) was calculated by: TV=0.5*a*b², where a was the long diameter of the tumor and b was the short diameter of the tumor.

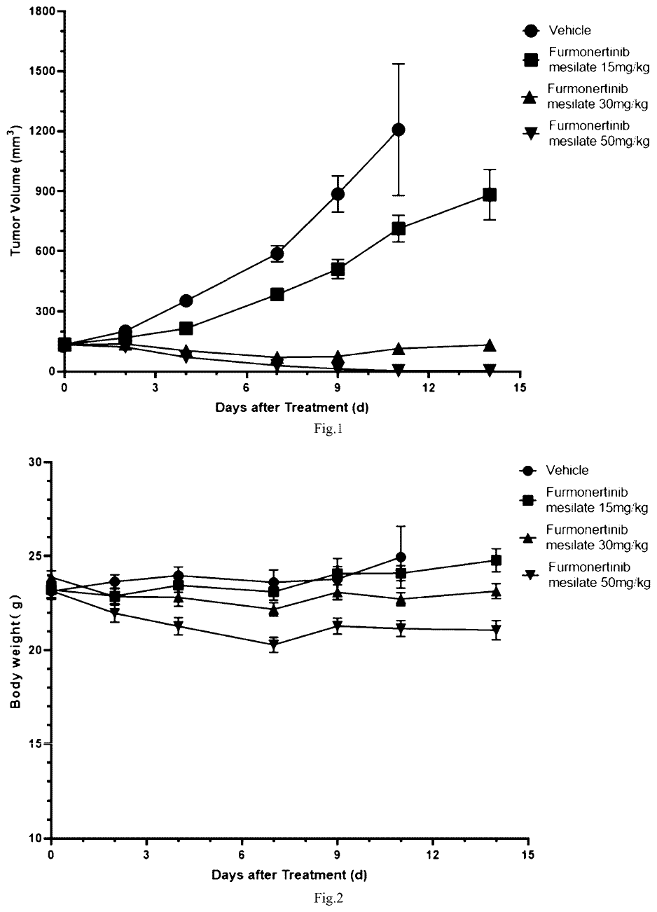

The curves for the tumor volume change of four experimental groups were shown in FIG. 1, and the curves for the body weight change of four experimental groups were shown in FIG. 2.

In summary, furmonertinib mesilate 15 mg/kg produced moderate anti-tumor activity; furmonertinib mesilate 30 mg/kg and furmonertinib mesilate 50 mg/kg produced extremely significant anti-tumor activity, and the three furmonertinib mesilate groups had little effect on the body weight of mice, and showed good safety.

### Test Example 3: Clinical Trial

An open-label, multi-center, dose-escalation and dose expansion study was designed and carried out to evaluate the safety, pharmacokinetics (PK), and preliminary antitumor activity of furmonertinib in patients with advanced or metastatic non-small cell lung cancer (NSCLC) with activating (including uncommon) Epidermal Growth Factor Receptor (EGFR) or Human Epidermal Growth Factor Receptor 2 (HER2) mutations. The first stage of the trial involved a dose escalation trial segment to evaluate once-daily dosing of furmonertinib starting at 240 mg daily, with subsequent patient cohorts to be administered furmonertinib at a higher or lower dose, if necessary, followed by expansion cohorts in defined patient groups with furmonertinib administered at 240 mg (and 160 mg in the case of EGFR PACC mutant NSCLC) daily.

General enrollment criteria include:
1) Histologically or cytologically documented, locally advanced or metastatic Non-Small Cell Lung Cancer (NSCLC) not amenable to curative surgery or radiotherapy;
2) Disease that has progressed after at least one available standard therapy; or for whom standard therapy has proven to be ineffective or intolerable; or for whom a clinical trial of an investigational agent is a recognized standard of care;
3) Documented radiologic disease progression during or after the last systemic anti-cancer therapy before the first dose of furmonertinib;
4) For patients with Epidermal Growth Factor Receptor (EGFR) mutations sensitive to osimertinib, the patient must have received osimertinib prior to study enrollment in regions where osimertinib is approved, including the US.

Exclusion criteria include:
1) Treatment with chemotherapy, targeted therapy, biologic therapy or an investigational agent as anti-cancer therapy within 3 or 5 elimination half-lives prior to initiation of furmonertinib, whichever is shorter;
2) Radiation therapy as cancer therapy within 4 weeks prior to initiation of furmonertinib;
3) Palliative radiation to bone metastases within 2 weeks prior to initiation of furmonertinib;
4) AE from prior anticancer therapy that have not resolved to Grade ≤1 except for alopecia or Grade ≤2 peripheral neuropathy.

Preliminary data from the clinical trial evaluating furmonertinib (240 mg QD and 160 mg QD) on NSCLC patients who had EGFR PACC mutation and had not taken any previous TKI treatment has shown a decrease in tumor size and clinical activity. The preliminary tumor assessment data from 3 PACC patients were listed in the table 2 below:

TABLE 2
Preliminary data of the clinical trial

| | | | Maximum |
|---|---|---|---|
| | | | SLD % |
| | EGFR | Prior Anti-tumor | change from |
| Dose | Mutation | Therapy | baseline |
| 160 mg QD | E709A + L858R | None | −40% |
| 240 mg QD | G719A | None | −23% |
| 240 mg QD | S768I + V769L | 1L: CARBOPLATIN + | −30% |
| | | PEMETREXED + | |
| | | PEMBROLIZUMAB | |

SLD: sum of longest diameter

### INDUSTRIAL APPLICABILITY

The present invention provides a pharmaceutical composition containing a therapeutically effective amount of furmonertinib or a pharmaceutically acceptable salt thereof and optionally a pharmaceutically acceptable carrier, use of furmonertinib or a pharmaceutically acceptable salt thereof, and said pharmaceutical composition in manufacture of a medicament for treating and/or preventing a disease mediated by PACC mutation. The present invention also provides a method of treating and/or preventing a disease mediated by PACC mutation, wherein a therapeutically effective amount of furmonertinib or a pharmaceutically acceptable salt thereof is administered to a patient. The pharmaceutical composition of the present invention shows an excellent therapeutic effect on disease mediated by PACC mutation (for example, non-small cell lung cancer (NSCLC)) with little side effects and excellent safety.

1. A pharmaceutical composition comprising a therapeutically effective amount of furmonertinib or a pharmaceutically acceptable salt thereof and optionally a pharmaceutically acceptable carrier.

2. The pharmaceutical composition of claim 1, wherein the pharmaceutically acceptable salt is mesilate salt.

3. The pharmaceutical composition of claim 1, wherein the content of furmonertinib or a pharmaceutically acceptable salt thereof is 80 mg-400 mg, such as 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg.

4. The pharmaceutical composition of claim 1, wherein the content of furmonertinib or a pharmaceutically acceptable salt thereof is 80 mg, 160 mg, 240 mg or 320 mg.

5. The pharmaceutical composition of claim 1, wherein the content of furmonertinib or a pharmaceutically acceptable salt thereof is 80 mg.

6. The pharmaceutical composition of claim 1, wherein the content of furmonertinib or a pharmaceutically acceptable salt thereof is 160 mg.

7. The pharmaceutical composition of claim 1, wherein the content of furmonertinib or a pharmaceutically acceptable salt thereof is 240 mg.

8. The pharmaceutical composition of claim 1, wherein the pharmaceutical composition is present in the formulation form of a tablet or a capsule.

9. The pharmaceutical composition of claim 8, wherein in each unit formulation of the pharmaceutical composition, the content of furmonertinib or a pharmaceutically acceptable salt thereof is 10 mg-400 mg, such as 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg.

10. The pharmaceutical composition of claim 8, wherein in each unit formulation of the pharmaceutical composition, the content of furmonertinib or a pharmaceutically acceptable salt thereof is 20 mg-320 mg.

11. The pharmaceutical composition of claim 8, wherein in each unit formulation of the pharmaceutical composition, the content of furmonertinib or a pharmaceutically acceptable salt thereof is 20 mg, 40 mg, 80 mg, 160 mg, 240 mg or 320 mg.

12. The pharmaceutical composition of claim 8, wherein in each unit formulation of the pharmaceutical composition, the content of furmonertinib or a pharmaceutically acceptable salt thereof is 40 mg.

13. The pharmaceutical composition of claim 1, which further comprises at least one second therapeutic agent.

14. The pharmaceutical composition of claim 13, wherein the second therapeutic agent is selected from chemotherapeutic drug, targeted antitumor drug, antibody drug and immunotherapeutic drug.

15. Use of a pharmaceutical composition comprising a therapeutically effective amount of furmonertinib or a pharmaceutically acceptable salt thereof, and optionally a pharmaceutically acceptable carrier in manufacture of a medicament for treating and/or preventing a disease mediated by PACC mutation.

16. Use of furmonertinib or a pharmaceutically acceptable salt thereof in manufacture of a medicament for treating and/or preventing a disease mediated by PACC mutation.

17. Use of furmonertinib or a pharmaceutically acceptable salt thereof in combination of at least one second therapeutic agent in manufacture of a medicament for treating and/or preventing a disease mediated by PACC mutation.

18. The use of claim 15, wherein the pharmaceutically acceptable salt is mesilate salt.

19. The use of claim 15, wherein the content of furmonertinib or a pharmaceutically acceptable salt thereof is 80 mg-400 mg, such as 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg.

20. The use of claim 15, wherein the content of furmonertinib or a pharmaceutically acceptable salt thereof is 80 mg, 160 mg, 240 mg, or 320 mg.

21. The use of claim 15, wherein the content of furmonertinib or a pharmaceutically acceptable salt thereof is 80 mg.

22. The use of claim 15, wherein the content of furmonertinib or a pharmaceutically acceptable salt thereof is 160 mg.

23. The use of claim 15, wherein the content of furmonertinib or a pharmaceutically acceptable salt thereof is 240 mg.

24. The use of claim 15, wherein the pharmaceutical composition is present in the formulation form of a tablet or a capsule.

25. The use of claim 24, wherein in each unit formulation, the content of furmonertinib or a pharmaceutically acceptable salt thereof is 10 mg-400 mg, such as 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg.

26. The use of claim 24, wherein in each unit formulation, the content of furmonertinib or a pharmaceutically acceptable salt thereof is 20 mg-320 mg.

27. The use of claim 24, wherein in each unit formulation, the content of furmonertinib or a pharmaceutically acceptable salt thereof is 20 mg, 40 mg, 80 mg, 160 mg, 240 mg or 320 mg.

28. The use of claim 24, wherein in each unit formulation, the content of furmonertinib or a pharmaceutically acceptable salt thereof is 40 mg.

29. The use of claim 15, wherein said pharmaceutical composition further comprises at least one second therapeutic agent.

30. The use of claim 29, wherein the second therapeutic agent is selected from chemotherapeutic drug, targeted antitumor drug, antibody drug and immunotherapeutic drug.

31. The use of claim 15, wherein the disease is cancer, such as lung cancer, such as non-small cell lung cancer (NSCLC).

32. The use of claim 15, wherein the disease is locally advanced non-small cell lung cancer or metastatic non-small cell lung cancer.

33. The use of claim 15, wherein the disease is a treatment-naive non-small cell lung cancer or a previously-treated non-small cell lung cancer.

34. The use of claim 15, wherein the PACC mutation is at least one selected from a group consisting of EGFR-G719S mutation, EGFR-S768I mutation, EGFR-G724S mutation, EGFR-L718Q mutation, EGFR-Del19/G724S mutation, EGFR-E709H mutation, EGFR-L747S mutation, EGFR-E709V mutation, EGFR-L747V mutation, EGFR-E709-710>D mutation, EGFR-E709A mutation, EGFR-Del19/C797S mutation, EGFR-L858R/C797S mutation, EGFR-L747S/G719A mutation, EGFR-L858R-L718V mutation, EGFR-L858R-L718Q mutation, EGFR-E709K-G719A mutation, EGFR-G719A mutation, EGFR-V769L mutation and EGFR-L861Q mutation.

35. The use of claim 15, wherein the PACC mutation is at least one selected from a group consisting of EGFR-G719S mutation, EGFR-S768I mutation, EGFR-G724S mutation, EGFR-Del19/G724S mutation, EGFR-E709H mutation, EGFR-L747S mutation, EGFR-E709V mutation, EGFR-L747V mutation, EGFR-E709-710>D mutation, EGFR-E709A mutation, EGFR-L747S/G719A mutation, EGFR-L858R-L718V mutation, EGFR-E709K-G719A mutation, EGFR-G719A mutation, EGFR-V769L mutation and EGFR-L861Q mutation.

36. A method of treating and/or preventing a disease mediated by PACC mutation, comprising administering to a patient a therapeutically effective amount of furmonertinib or a pharmaceutically acceptable salt thereof.

37. A method of treating and/or preventing a disease comprising administering to a patient with positive PACC mutation a therapeutically effective amount of furmonertinib or a pharmaceutically acceptable salt thereof.

38. A method of treating locally advanced or metastatic non-small cell lung cancer comprising administering to a patient in need thereof a therapeutically effective amount of furmonertinib or a pharmaceutically acceptable salt thereof.

39. A method of treating locally advanced or metastatic non-small cell lung cancer comprising administering to a patient with confirmed positive PACC mutation a therapeutically effective amount of furmonertinib or a pharmaceutically acceptable salt thereof.

40. A method of treating locally advanced or metastatic non-small cell lung cancer comprising administering to a patient harboring PACC mutation a therapeutically effective amount of furmonertinib or a pharmaceutically acceptable salt thereof.

41. A method of treating locally advanced or metastatic non-small cell lung cancer comprising administering to a patient with confirmed positive PACC mutation who has received no prior systematic anti-tumor therapy a therapeutically effective amount of furmonertinib or a pharmaceutically acceptable salt thereof.

42. A method of treating locally advanced or metastatic non-small cell lung cancer comprising administering to a patient with confirmed positive PACC mutation who has progressive disease after receiving prior systematic anti-tumor therapy a therapeutically effective amount of furmonertinib or a pharmaceutically acceptable salt thereof.

43. The method of claim 36, comprising administering furmonertinib or a pharmaceutically acceptable salt thereof at a dose of 80 mg-400 mg, such as 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg.
